(19) United States
(12) Patent Application Publication
(10) Pub. No.: US 2026/0285913 A1
(43) Pub. Date: Sep. 24, 2026

(54) METHODS OF PRODUCING HIV-1 ENVELOPE GLYCOPROTEINS AND GUANOSINE MICROPARTICLES THEREOF

(71) Applicants: Jose A. LASALDE-DOMINICCI, San Juan, PR (US); Manuel DELGADO-VELEZ, San Juan, PR (US); Jose RIVERA, San Juan, PR (US); Luis PRIETO-COSTAS, San Juan, PR (US); PUERTO RICO SCIENCE TECHNOLOGY & RESEARCH TRUST, San Juan, PR (US)

(72) Inventors: Jose A. LASALDE-DOMINICCI, San Juan, PR (US); Manuel DELGADO-VELEZ, San Juan, PR (US); Jose RIVERA, San Juan, PR (US); Luis PRIETO-COSTAS, San Juan, PR (US)

(21) Appl. No.: 19/475,469

(22) PCT Filed: Apr. 15, 2024
(86) PCT No.: PCT/US2024/024602

§ 371 Date: Oct. 15, 2025

Related U.S. Application Data
(60) Provisional application No. 63/459,620, filed on Apr. 15, 2023.

(51) Int. Cl. C07K 14/005; A61K 39/00

(52) U.S. Cl. CPC C07K 14/005; A61K 39/00; A61K 2039/55555; C12N 2740/16051; C12N 2740/16122; C12N 2740/16134

(57) ABSTRACT

The present disclosure provides methods of producing HIV envelope glycoproteins such as gp145 that provide higher levels of protein production and higher titers compared to current methods. Moreover, the present disclosure provides guanosine microparticles capable of encapsulating and presenting HIV envelope glycoproteins such as gp145 as antigens. These microparticles can be utilized for treatment and/or of HIV in patients in need thereof.

### CROSS-REFERENCE TO RELATED APPLICATIONS

This application claims the benefit under 35 U.S.C. § 119(e) of U.S. Provisional Application Ser. No. 63/459,620, filed on Apr. 15, 2023, the entire disclosures of which is incorporated herein by reference.

### BACKGROUND AND SUMMARY

Despite tremendous research efforts and prevention campaigns, the human immunodeficiency virus (HIV) remains a costly global health threat. As of 2020, 38 mil-lion individuals were living with HIV with 1.5 million new infections, 160,000 of them children under the age of fourteen years. Although the number of new infections has decreased by 25% since 2010, lowering the incidence of HIV globally will likely require the development and deployment of a vaccine.

The surface envelope glycoprotein (Env) of the HIV has been the primary target for vaccine development during last decades. It is made by the virus as a single polypeptide, gp160, which is cleaved into gp120 and gp41 to form the mature protein. The uncleaved trimeric Env is also referred to as gp145.

An important obstacle to large-scale production of longer versions of the Env glycoprotein is manufacturability. Although the soluble gp120 can provide higher yields, the longer Env construct containing portions of the transmembrane gp41 are known to be problematic and give lower yields. In particular, the production titers of recombinant Env are generally very low. It has been estimated that the titers for Env glycoproteins are about 100-fold lower (~10 mg/L) than the titers for the production of monoclonal antibodies using mammalian cells. Accordingly, there exists a need for new methods of producing HIV envelope glycoproteins such as gp145. Furthermore, new compositions comprising gp145 that could be utilized as immunogenic means are also highly desirable.

Accordingly, the present disclosure provides methods of producing HIV envelope glycoproteins such as gp145 that provide higher levels of protein production and higher titers compared to current methods. Moreover, the present disclosure provides guanosine microparticles capable of encapsulating and presenting HIV envelope glycoproteins such as gp145 as antigens. These microparticles can be utilized for treatment and/or of HIV in patients in need thereof. Advantageously, the methods of the present disclosure provide a four-fold improvement in titer of gp145 protein while ensuring that the quality attributes for gp145 was maintained. Importantly, the methods described herein are transferable to a cGMP-regulated manufacturing facility.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTIONS OF THE DRAWINGS

The detailed description particularly refers to the accompanying figures in which:

FIGS. 1A-1D show the pH effects on CHO-K1 cell growth. Cells were grown in a 0.250 L bioreactor at different pH set points. FIG. 1A shows an assessment of bioreactor viable cell density (VCD) of cells grown at different pHs. FIG. 1B shows the percent viability. FIG. 1C shows the glucose consumption profile. FIG. 1D shows the lactate production/consumption of cells grown at different pHs. The data presented are the average of two runs and the error bars represent the standard deviation. On days 10 and 11, a single bioreactor was left until day 11. The shake flask (SF) was run as a control (black). In all figures, Red=pH 7.2; Blue=pH 7.0; Green=pH 6.8.

FIGS. 2A-2C show an analysis of metabolites present in cell cultures during pH studies in 0.250 L bioreactors. FIG. 2A shows the glutamine levels, FIG. 2B shows GlutaMAX concentration, and FIG. 2C shows the ammonia profile. A Cedex Bio Analyzer was employed to measure metabolites from supernatants. In each figure the following colors are used: black for control shake flask (SF); red for pH 7.2; blue for pH 7.0; green for pH 6.8.

FIGS. 3A-3D show Production of gp145 in a 40-L bioreactor. FIG. 3A shows an assessment of VCD and percent viability over the 11 days from seeding to harvest for the growth. FIG. 3B shows the daily titer determination by RP-HPLC and CHO host cell proteins (HCPs) quantification. The titer values are the average of three readings. The HCP values are an average of 12 readings. In both graphs, the error shown is the standard deviation. In FIG. 3C shown is a profile of other metabolites for the growth (red=lactate; orange=ammonia, (NH₃); green=glutamate; blue=GlutaMAX; purple=glutamine; dashed line=lactate dehydrogenase, LDH). FIG. 3D shows the glucose profile of the growth. The lower detection limits of the instrument are: glucose (0.02 g/L), glutamate (0.1 mmol/L), glutamine (0.01 mmol/L), GlutaMAX (0.01 mmol/L), NH₃ (0.278 mmol/L), lactate (0.0444 mmol/L), and LDH (20 U/L).

FIGS. 4A-4C show the molecular mass determination of HIV gp145 by SDS-PAGE, MALDI-TOF, and MCGE. The gp145 from the 50-L bioreactor was analyzed. As shown in FIG. 4A, a SDS-PAGE analysis, 10 μg of gp145 produced in CHO cells was applied. FIG. 4B shows the MALDI-TOF whole protein analysis was performed using the CHCA matrix in linear positive mode. FIG. 4C shows the MCGE analysis of the gp145 (7 ng) was carried out with the High Sensitivity Protein 250 kit, to produce a gel-like image representative of the run. In panel C, the M column represents protein markers. RM=reference standard.

FIGS. 5A-5B show protein purity analysis by microchip capillary gel electrophoresis. FIG. 5A shows an electropherogram coming from the MCGE analysis shown in FIG. 4C for the reference standard (RM). The largest peak represents the RM, exhibiting a purity of 90%. The other small peaks represent impurities. FIG. 5B shows an electropherogram from the MCGE analysis shown in FIG. 4C for gp145. The largest peak represents gp145, which is 78% pure. Green peaks represent the lower marker peak, which includes a fluorescent dye and excess labeling dye. The Y-axis represents fluorescence units (FU) while the X-axis is time in seconds.

FIGS. 6A-6B show peptide mass fingerprinting (PMF) of gp145 Env. Where FIG. 6A shows that RM and gp145 have similar PMF profiles. Proteins were not treated with PNGase F. FIG. 6B shows PNGase F treatment (right) increases the PMF coverage relative to untreated (PNGase F⁻) sample (left). Ten micrograms of RM was used for analysis. The gp145 was resolved in SDS-PAGE and then excised followed by PNGase F treatment (where indicated), prior to trypsin digestion. Peptides were co-crystallized with α-cyano-4-hydroxycinnamic acid (CHCA) matrix and flown in reflector positive mode on the MALDI-TOF.

FIG. 7 shows glycan analysis of the N-linked glycans released from HIV gp145 protein. This glycan profile shows the relative abundance of the most abundant neutral glycans identified by positive reflector MALDI-TOF analysis. The recombinant gp145 (10 μg) was resolved through SDS-PAGE gel and digested with PNGase F. Recovered N-glycans were co-crystalized with THAP matrix. The predicted glycan structures are shown, from left to right, oligomannose-5, oligomannose-6, oligomannose-7, oligomannose-8, G2F, and oligomannose-9. Error bars represent SEM. RM=reference standard.

FIGS. 8A-8B show sample data of N-linked glycans identified by MALDI. FIG. 8A shows Positive Ion MALDI-TOF analysis of neutral N-glycans released from HIV gp145 protein RM. The recombinant gp145 (10 μg) was resolved in a SDS-PAGE and digested with PNGase F. Recovered N-glycans were co-crystalized with THAP matrix. FIG. 8B shows Negative Ion MALDI-TOF analysis of acidic N-glycans released from HIV gp145 protein RM. The sample HIV gp145 N-glycans from FIG. 8A were used for analysis in the reflector negative mode. Neuraminidase A treatment confirms the glycans which are sialylated. The predicted glycan structures are shown and the mass of the glycans is expressed as m/z in both figures. Mannose (Man), galactose (Gal), fucose (Fuc), N-acetyl glucosamine (GlcNAc), and N-acetyl neuraminic acid or sialic acid (NeuNAc).

FIGS. 9A-9B show the biochemical characterization of gp145. Specifically, FIG. 9A shows a representative glycoform pI profile by cIEF of HIV gp145 protein. Buffer was used as a blank control. The pI markers 3.59 and 9.46 were used to calibrate the electropherograms. FIG. 9B shows a representative trace of the RP-HPLC hydrophobic footprint analysis of gp145 Env. RM=reference standard.

FIG. 10 shows hydrophobic footprint analysis of gp145 Env. The standard curved employed to calculate the percent recovery. Error on the graph is shown as standard deviation.

FIGS. 11A-11B show size analysis of gp145 by dynamic light scattering (DLS). FIG. 11A shows RM gp145 protein analysis via DLS, demonstrating a size of about 23.5±2 d.nm. FIG. 11B shows gp145 analysis using DLS, which demonstrates a size around 21.5±1.5 d.nm. The three curves in each histogram represent three individual measurements. X-axis is in log scale. RM=reference standard.

FIG. 12 shows the oligomerization analysis of gp145. The gp145, 10 μg, (lanes 2 and 3) analysis by native PAGE. The NativeMark (NM) protein standard was used in lane 1 (Invitrogen Novex). RM=reference standard.

FIG. 13 shows, on the left, a chromatogram profile of purified gp145; the right chromatogram is that of the reference material (RM). The arrow indicates likely higher order aggregates. The peak numbers indicate fractions that were collected and analyzed by native gel (FIG. 14). This analysis was performed using an AKTA FPLC system and a Superdex 200 Increase 10/300 GL column (Cytiva) with 1×PBS as the mobile phase (Sigma tablet, P4417-100TAB). The parameters used for the gp145 run were as follows: 0.15 ml/min flow rate, 50 μl injection volume, and 0.34 mg/ml protein concentration. The parameters for the RM run were: 0.7 ml/min flow rate, 100 μl injection volume, and 1 mg/ml protein concentration.

FIG. 14 shows the native gel analysis of RM SEC peaks. Where the image shows the 10% TGX precast native PAGE gel (Bio-Rad). The gel was loaded as follows: lane 3, peak 1; lane 4, peak 2; lane 5; peak 3 and lane 7, SEC load.

FIGS. 15A-15B shows the integrity analysis of gp145. FIG. 15A shows the CD analysis of gp145. The protein was diluted in 0.1×D-PBS. RM=reference standard. FIG. 15B shows Bio-layer interferometry analysis of gp145. The gp145 binds the broadly neutralizing antibody, 4E10, with an affinity of 65 nM.

FIG. 16 shows the gp145 RM binding affinity analysis. The RM gp145 binds the broadly neutralizing antibody, 4E10, with a binding affinity of 46 nM. BLI sensorgram generated by binding of serial dilutions of gp145. Black lines represent the 1:1 global fit curves. RM=reference standard.

FIGS. 17A-17D show the encapsulation of gp145 in SHS microparticles. FIG. 17A specifically shows microparticles that were evaluated by flow cytometry where the gp120-FITC glycoprotein was incubated and encapsulated in the microparticles (lower panels). FIG. 17B shows that the observed encapsulation was statistically significant. FIG. 17C shows microparticles which were evaluated by flow cytometry before being used to encapsulate the antigen (top panels). In the lower panels, microparticles containing gp145-FITC show a right shift toward higher values of fluorescence. FIG. 17D shows that encapsulation of gp145-FITC was effectively achieved as determined statistically. ****P<0.0001, unpaired t test.

FIGS. 18A-18F show that SHS microparticles encapsulate and present gp145 on their surface. FIG. 18A shows a representative gp145-FITC-containing microparticle on the left. The lack of Alexa Fluor 647 signal in the middle panel microparticle indicates that there is no autofluorescence at the analyzed wavelength. A representative DIC image of microparticles is shown on the right. FIG. 18B shows the fluorescence emitted by the encapsulated gp145-FITC as depicted in 2D in the extreme left panel, the absence of fluorescence at the analyzed wavelength in the central panel, and the 2D DIC image in the extreme right panel. FIG. 18C shows a graph depicting the fluorescence emitted by gp145-FITC encapsulated (green line). In DIC, the microparticle is represented by a blue line with blue arrows indicating its borders. FIG. 18D shows a three-dimensional representation of a microparticle encapsulating gp145-FITC (left panel). The binding of a secondary antibody conjugated to Alexa Fluor 647 that recognizes VRC01 and strongly binds to gp145-FITC is shown in the center panel. A representative 3D DIC image is shown (right panel). FIG. 18E shows a representative 2D image of encapsulated gp145-FITC is shown (far left panel). In the middle panel, VRC01 recognizes gp145-FITC fluorescence, while DIC capture is shown in the far right panel. FIG. 18F shows a graph of encapsulated gp145-FITC (green line), gp145-FITC on the microparticle surface (red line), and the DIC corresponding to the microparticle (blue line).

FIGS. 19A-19D show that the antibody against VRC01 has negligible binding towards the SHS microparticles. FIG. 19A shows in the upper 3D panels, from left to right, that the FITC signal is not detected in microparticles lacking gp145-FITC (far left panel). As can be seen, the secondary antibody exhibits some affinity for the microparticle (middle panel). On the far right panel, the DIC representation of the microparticles is shown. FIG. 19B shows 2D confocal images demonstrating the absence of FITC detection (far left panel), weak signal detection of Alexa Fluor 647 (middle panel), and DIC representation (far right panel). FIG. 19C shows a plot of the DIC borders of the microparticle (blue arrows) and the fluorescence emanating from the secondary antibody recognizing VRC01. Note that the detection of the latter is below the detection of the DIC (red arrows). FIG. 19D shows that although there is some antibody binding to the microparticle, the magnitude of fluorescence after gp145-FITC encapsulation significantly exceeds (*P=0.0348) non-specific binding of the antibody. There is no incorporation of the antibody since its signal (Alexa Fluor 647) overlaps with the FITC signal, a fluorophore absent in the experiment (panels A and B on the extreme left). Statistical analysis employed was an unpaired t test.

FIG. 20 shows a summary of the scale-up capabilities and increased titers of gp145. Technology transfer 5 L bioreactor reported titers, blue; 1 L bioreactor titers, orange (n=7); 5 L bioreactor titers, light blue (n=2); 10 L bioreactor titer, dark blue (n=5); 40 L bioreactor titer, grey (n=1); 1 L glucose feed, yellow (n=5); 1 L glucose feed plus GlutaMAX, brown (n=1).

### DETAILED DESCRIPTION

Various embodiments of the invention are described herein as follows.

In an illustrative aspect, a method of producing a protein is provided. The method comprises the step of culturing one or more cells comprising the protein, wherein the cells express the protein.

In an embodiment, the culturing is performed in a cell culture medium, wherein the cells secrete the protein into the cell culture medium. In an embodiment, the method further comprises a step of obtaining the protein from the cell culture medium.

In an embodiment, the method further comprises a step of harvesting the cells. In an embodiment, the method further comprises a step of lysing the harvested cells. In an embodiment, the method further comprises a step of separating protein from the lysed cells.

In an embodiment, the method comprises isolating the protein from the cells. In an embodiment, the protein is an HIV-1 envelope glycoprotein. In an embodiment, the protein is gp145.

In an embodiment, the cells are Chinese Hamster Ovary (CHO) cells. In an embodiment, the CHO cells are CHO-K1 cells. In an embodiment, the CHO cells are engineered cells. In an embodiment, the engineered cells express gp145. In an embodiment, gp145 is uncleaved. In an embodiment, the engineered cells overexpress gp145. In an embodiment, gp145 is uncleaved.

In an embodiment, the culturing is performed in a cell culture medium. In an embodiment, the cell culture medium comprises glucose. In an embodiment, the cell culture medium maintains glucose at a range between 2 g/L and 3 g/L. In an embodiment, the cell culture medium maintains glucose at a range between 3 g/L and 4 g/L. In an embodiment, the cell culture medium maintains glucose at a range between 4 g/L and 5 g/L. In an embodiment, the cell culture medium maintains glucose at a range between 5 g/L and 6 g/L. In an embodiment, the cell culture medium maintains glucose at a range between 4 g/L and 6 g/L.

In an embodiment, the cell culture medium comprises glutamine. In an embodiment, the cell culture medium comprises L-glutamine. In an embodiment, the cell culture medium comprises L-alanyl-L-glutamine.

In an embodiment, the cell culture medium comprises penicillin. In an embodiment, the cell culture medium comprises streptomycin. In an embodiment, the cell culture medium comprises puromycin.

In an embodiment, the cell culture medium receives continuous feeding of glucose. As described herein, continuous feeding refers to administration of a component at a specified rate and/or time, based on evaluation of one or more parameters of the cell culturing (e.g., cell growth, cell density, and the like). In an embodiment, the cell culture medium receives continuous feeding of glutamine. In an embodiment, the cell culture medium receives continuous feeding of L-glutamine. In an embodiment, the cell culture medium receives continuous feeding of L-alanyl-L-glutamine.

In an embodiment, the culturing is performed at a pH between 6.0 and 8.0. In an embodiment, the pH is between 6.2 and 7.8. In an embodiment, the pH is between 6.4 and 7.6.

In an embodiment, the pH is between 6.6 and 7.4. In an embodiment, the pH is between 6.8 and 7.2.

In an embodiment, the culturing is performed at a pH of 6.1. In an embodiment, the culturing is performed at a pH of 6.2. In an embodiment, the culturing is performed at a pH of 6.3. In an embodiment, the culturing is performed at a pH of 6.4. In an embodiment, the culturing is performed at a pH of 6.5. In an embodiment, the culturing is performed at a pH of 6.6. In an embodiment, the culturing is performed at a pH of 6.7. In an embodiment, the culturing is performed at a pH of 6.8. In an embodiment, the culturing is performed at a pH of 6.9. In an embodiment, the culturing is performed at a pH of 7.0. In an embodiment, the culturing is performed at a pH of 7.1. In an embodiment, the culturing is performed at a pH of 7.2. In an embodiment, the culturing is performed at a pH of 7.3. In an embodiment, the culturing is performed at a pH of 7.4. In an embodiment, the culturing is performed at a pH of 7.5. In an embodiment, the culturing is performed at a pH of 7.6. In an embodiment, the culturing is performed at a pH of 7.7. In an embodiment, the culturing is performed at a pH of 7.8. In an embodiment, the culturing is performed at a pH of 7.9.

In an embodiment, the culturing is performed at a temperature between 30° C. and 40° C. In an embodiment, the culturing is performed at a temperature between 32° C. and 38° C. In an embodiment, the culturing is performed at a temperature between 34° C. and 38° C. In an embodiment, the culturing is performed at a temperature between 34° C. and 36° C. In an embodiment, the culturing is performed at a temperature between 36° C. and 38° C.

In an embodiment, the culturing is performed at a temperature of 30° C. In an embodiment, the culturing is performed at a temperature of 31° C. In an embodiment, the culturing is performed at a temperature of 32° C. In an embodiment, the culturing is performed at a temperature of 33° C. In an embodiment, the culturing is performed at a temperature of 34° C. In an embodiment, the culturing is performed at a temperature of 35° C. In an embodiment, the culturing is performed at a temperature of 36° C. In an embodiment, the culturing is performed at a temperature of 37° C. In an embodiment, the culturing is performed at a temperature of 38° C. In an embodiment, the culturing is performed at a temperature of 39° C. In an embodiment, the culturing is performed at a temperature of 40° C.

In an embodiment, the culturing is performed at a global oxygen delivery between 25% and 75%. In an embodiment, the culturing is performed at a global oxygen delivery between 25% and 50%. In an embodiment, the culturing is performed at a global oxygen delivery between 35% and 50%. In an embodiment, the culturing is performed at a global oxygen delivery of 25%. In an embodiment, the culturing is performed at a global oxygen delivery of 30%. In an embodiment, the culturing is performed at a global oxygen delivery of 35%. In an embodiment, the culturing is performed at a global oxygen delivery of 40%. In an embodiment, the culturing is performed at a global oxygen delivery of 45%. In an embodiment, the culturing is performed at a global oxygen delivery of 50%. In an embodiment, the culturing is performed at a global oxygen delivery of 55%. In an embodiment, the culturing is performed at a global oxygen delivery of 60%. In an embodiment, the culturing is performed at a global oxygen delivery of 65%. In an embodiment, the culturing is performed at a global oxygen delivery of 70%. In an embodiment, the culturing is performed at a global oxygen delivery of 75%.

In an embodiment, the culturing is performed at an air oxygen flow of 1 SLPH.

In an embodiment, the culturing is performed at an agitation between 50 rpm and 150 rpm. In an embodiment, the culturing is performed at an agitation between 60 rpm and 120 rpm. In an embodiment, the culturing is performed at an agitation between 60 rpm and 90 rpm. In an embodiment, the culturing is performed at an agitation between 90 rpm and 120 rpm. In an embodiment, the culturing is performed at an agitation of 50 rpm. In an embodiment, the culturing is performed at an agitation of 60 rpm. In an embodiment, the culturing is performed at an agitation of 70 rpm. In an embodiment, the culturing is performed at an agitation of 80 rpm. In an embodiment, the culturing is performed at an agitation of 90 rpm. In an embodiment, the culturing is performed at an agitation of 100 rpm. In an embodiment, the culturing is performed at an agitation of 110 rpm. In an embodiment, the culturing is performed at an agitation of 120 rpm. In an embodiment, the culturing is performed at an agitation of 130 rpm. In an embodiment, the culturing is performed at an agitation of 140 rpm. In an embodiment, the culturing is performed at an agitation of 150 rpm.

In an embodiment, the culturing is performed at a seeding density of 0.4×10⁶ cells per mL. In an embodiment, the culturing is performed at a seeding density of 0.5×10⁶ cells per mL. In an embodiment, the culturing is performed at a seeding density of 0.6×10⁶ cells per mL. In an embodiment, the culturing is performed at a seeding density of 0.7×10⁶ cells per mL. In an embodiment, the culturing is performed at a seeding density of 0.8×10⁶ cells per mL. In an embodiment, the culturing is performed at a seeding density of 0.9×10⁶ cells per mL. In an embodiment, the culturing is performed at a seeding density of 1.0×10⁶ cells per mL.

In an embodiment, the cell culturing provides a viable cell density (VCD) above 98%. In an embodiment, the cell culturing provides a VCD above 95%. In an embodiment, the cell culturing provides a VCD above 90%. In an embodiment, the cell culturing provides a VCD above 85%. In an embodiment, the cell culturing provides a VCD above 80%. In an embodiment, the cell culturing provides a VCD above 75%. In an embodiment, the cell culturing provides a VCD above 70%. In an embodiment, the cell culturing provides a VCD above 65%. In an embodiment, the cell culturing provides a VCD above 60%. In an embodiment, the cell culturing provides a VCD above 55%. In an embodiment, the cell culturing provides a VCD above 50%. In an embodiment, the cell culturing provides a VCD above 45%. In an embodiment, the cell culturing provides a VCD above 40%.

In an embodiment, the method provides an increased expression level of the protein. In an embodiment, the method provides an increased titer level of the protein. In an embodiment, the method provides an increased amount of production of the protein.

In an illustrative aspect, a guanosine microparticle comprising a protein is provided. Guanosine microparticles are described in U.S. Pat. No. 10,106,572, which is hereby incorporated by reference in its entirety.

In an embodiment, the protein is an HIV-1 envelope glycoprotein. In an embodiment, the protein is gp145. In an embodiment, the protein is encapsulated in the microparticle. In an embodiment, the protein is presented on the outer surface of the microparticle.

In an embodiment, the microparticle has a size between 1000 nm and 2000 nm. In an embodiment, the microparticle has a size between 1000 nm and 1100 nm. In an embodiment, the microparticle has a size between 1100 nm and 1200 nm. In an embodiment, the microparticle has a size between 1200 nm and 1300 nm. In an embodiment, the microparticle has a size between 1300 nm and 1400 nm. In an embodiment, the microparticle has a size between 1400 nm and 1500 nm. In an embodiment, the microparticle has a size between 1500 nm and 1600 nm. In an embodiment, the microparticle has a size between 1600 nm and 1700 nm. In an embodiment, the microparticle has a size between 1700 nm and 1800 nm. In an embodiment, the microparticle has a size between 1800 nm and 1900 nm. In an embodiment, the microparticle has a size between 1900 nm and 2000 nm.

In an embodiment, the microparticle is an adjuvant. In an embodiment, the microparticle is configured for presentation of an antigen. In an embodiment, the microparticle is configured for delivery of a therapeutic agent. In an embodiment, the therapeutic agent is encapsulated in the microparticle.

In an illustrative aspect, a method of delivering a therapeutic agent to a patient is provided. The method comprises the step of administering a guanosine microparticle as described herein to a patient. In an embodiment, the microparticle comprises the therapeutic agent.

In an illustrative aspect, a method of presenting an antigen to a patient is provided. The method comprises the step of administering a guanosine microparticle as described herein to a patient. In an embodiment, the microparticle comprises the antigen.

In an illustrative aspect, a method of administering an adjuvant to a patient is provided. The method comprises the step of administering a guanosine microparticle as described herein to a patient. In an embodiment, the microparticle is the adjuvant.

The following numbered embodiments are contemplated and are non-limiting:
1. A method of producing a protein, the method comprising the step of culturing one or more cells comprising the protein, wherein the cells express the protein.
2. The method of clause 1, any other suitable clause, or any combination of suitable clauses, wherein the culturing is performed in a cell culture medium and wherein the cells secrete the protein into the cell culture medium.
3. The method of clause 2, any other suitable clause, or any combination of suitable clauses, wherein the method further comprises a step of obtaining the protein from the cell culture medium.
4. The method of clause 1, any other suitable clause, or any combination of suitable clauses, wherein the method further comprises a step of harvesting the cells.
5. The method of clause 4, any other suitable clause, or any combination of suitable clauses, wherein the method further comprises a step of lysing the harvested cells.
6. The method of clause 5, any other suitable clause, or any combination of suitable clauses, wherein the method further comprises a step of separating protein from the lysed cells.
7. The method of clause 1, any other suitable clause, or any combination of suitable clauses, wherein the method comprises isolating the protein from the cells.
8. The method of clause 1, any other suitable clause, or any combination of suitable clauses, wherein the protein is an HIV-1 envelope glycoprotein.
9. The method of clause 1, any other suitable clause, or any combination of suitable clauses, wherein the protein is gp145.
10. The method of clause 1, any other suitable clause, or any combination of suitable clauses, wherein the cells are Chinese Hamster Ovary (CHO) cells.
11. The method of clause 10, any other suitable clause, or any combination of suitable clauses, wherein the CHO cells are CHO-K1 cells.
12. The method of clause 10, any other suitable clause, or any combination of suitable clauses, wherein the CHO cells are engineered cells.
13. The method of clause 12, any other suitable clause, or any combination of suitable clauses, wherein the engineered cells express gp145.
14. The method of clause 13, any other suitable clause, or any combination of suitable clauses, wherein gp145 is uncleaved.
15. The method of clause 12, any other suitable clause, or any combination of suitable clauses, wherein the engineered cells overexpress gp145.
16. The method of clause 15, any other suitable clause, or any combination of suitable clauses, wherein gp145 is uncleaved
17. The method of clause 1, any other suitable clause, or any combination of suitable clauses, wherein the culturing is performed in a cell culture medium.
18. The method of clause 17, any other suitable clause, or any combination of suitable clauses, wherein the cell culture medium comprises glucose.
19. The method of clause 17, any other suitable clause, or any combination of suitable clauses, wherein the cell culture medium maintains glucose at a range between 2 g/L and 3 g/L.
20. The method of clause 17, any other suitable clause, or any combination of suitable clauses, wherein the cell culture medium maintains glucose at a range between 3 g/L and 4 g/L.
21. The method of clause 17, any other suitable clause, or any combination of suitable clauses, wherein the cell culture medium maintains glucose at a range between 4 g/L and 5 g/L.
22. The method of clause 17, any other suitable clause, or any combination of suitable clauses, wherein the cell culture medium maintains glucose at a range between 5 g/L and 6 g/L.
23. The method of clause 17, any other suitable clause, or any combination of suitable clauses, wherein the cell culture medium maintains glucose at a range between 4 g/L and 6 g/L.
24. The method of clause 17, any other suitable clause, or any combination of suitable clauses, wherein the cell culture medium comprises glutamine.
25. The method of clause 17, any other suitable clause, or any combination of suitable clauses, wherein the cell culture medium comprises L-glutamine.
26. The method of clause 17, any other suitable clause, or any combination of suitable clauses, wherein the cell culture medium comprises L-alanyl-L-glutamine.
27. The method of clause 17, any other suitable clause, or any combination of suitable clauses, wherein the cell culture medium comprises penicillin.
28. The method of clause 17, any other suitable clause, or any combination of suitable clauses, wherein the cell culture medium comprises streptomycin.
29. The method of clause 17, any other suitable clause, or any combination of suitable clauses, wherein the cell culture medium comprises puromycin.
30. The method of clause 17, any other suitable clause, or any combination of suitable clauses, wherein the cell culture medium receives continuous feeding of glucose.
31. The method of clause 17, any other suitable clause, or any combination of suitable clauses, wherein the cell culture medium receives continuous feeding of glutamine.
32. The method of clause 17, any other suitable clause, or any combination of suitable clauses, wherein the cell culture medium receives continuous feeding of L-glutamine.
33. The method of clause 17, any other suitable clause, or any combination of suitable clauses, wherein the cell culture medium receives continuous feeding of L-alanyl-L-glutamine.
34. The method of clause 1, any other suitable clause, or any combination of suitable clauses, wherein the culturing is performed at a pH between 6.0 and 8.0.
35. The method of clause 1, any other suitable clause, or any combination of suitable clauses, wherein the pH is between 6.2 and 7.8.
36. The method of clause 1, any other suitable clause, or any combination of suitable clauses, wherein the pH is between 6.4 and 7.6.
37. The method of clause 1, any other suitable clause, or any combination of suitable clauses, wherein the pH is between 6.6 and 7.4.
38. The method of clause 1, any other suitable clause, or any combination of suitable clauses, wherein the pH is between 6.8 and 7.2.
39. The method of clause 1, any other suitable clause, or any combination of suitable clauses, wherein the culturing is performed at a pH of 6.1.
40. The method of clause 1, any other suitable clause, or any combination of suitable clauses, wherein the culturing is performed at a pH of 6.2.
41. The method of clause 1, any other suitable clause, or any combination of suitable clauses, wherein the culturing is performed at a pH of 6.3.
42. The method of clause 1, any other suitable clause, or any combination of suitable clauses, wherein the culturing is performed at a pH of 6.4.
43. The method of clause 1, any other suitable clause, or any combination of suitable clauses, wherein the culturing is performed at a pH of 6.5.
44. The method of clause 1, any other suitable clause, or any combination of suitable clauses, wherein the culturing is performed at a pH of 6.6.
45. The method of clause 1, any other suitable clause, or any combination of suitable clauses, wherein the culturing is performed at a pH of 6.7.
46. The method of clause 1, any other suitable clause, or any combination of suitable clauses, wherein the culturing is performed at a pH of 6.8.
47. The method of clause 1, any other suitable clause, or any combination of suitable clauses, wherein the culturing is performed at a pH of 6.9.
48. The method of clause 1, any other suitable clause, or any combination of suitable clauses, wherein the culturing is performed at a pH of 7.0.
49. The method of clause 1, any other suitable clause, or any combination of suitable clauses, wherein the culturing is performed at a pH of 7.1.
50. The method of clause 1, any other suitable clause, or any combination of suitable clauses, wherein the culturing is performed at a pH of 7.2.
51. The method of clause 1, any other suitable clause, or any combination of suitable clauses, wherein the culturing is performed at a pH of 7.3.
52. The method of clause 1, any other suitable clause, or any combination of suitable clauses, wherein the culturing is performed at a pH of 7.4.
53. The method of clause 1, any other suitable clause, or any combination of suitable clauses, wherein the culturing is performed at a pH of 7.5.
54. The method of clause 1, any other suitable clause, or any combination of suitable clauses, wherein the culturing is performed at a pH of 7.6.
55. The method of clause 1, any other suitable clause, or any combination of suitable clauses, wherein the culturing is performed at a pH of 7.7.
56. The method of clause 1, any other suitable clause, or any combination of suitable clauses, wherein the culturing is performed at a pH of 7.8.
57. The method of clause 1, any other suitable clause, or any combination of suitable clauses, wherein the culturing is performed at a pH of 7.9.
58. The method of clause 1, any other suitable clause, or any combination of suitable clauses, wherein the culturing is performed at a temperature between 30° C. and 40° C.
59. The method of clause 1, any other suitable clause, or any combination of suitable clauses, wherein the culturing is performed at a temperature between 32° C. and 38° C.
60. The method of clause 1, any other suitable clause, or any combination of suitable clauses, wherein the culturing is performed at a temperature between 34° C. and 38° C.
61. The method of clause 1, any other suitable clause, or any combination of suitable clauses, wherein the culturing is performed at a temperature between 34° C. and 36° C.
62. The method of clause 1, any other suitable clause, or any combination of suitable clauses, wherein the culturing is performed at a temperature between 36° C. and 38° C.
63. The method of clause 1, any other suitable clause, or any combination of suitable clauses, wherein the culturing is performed at a temperature of 30° C.
64. The method of clause 1, any other suitable clause, or any combination of suitable clauses, wherein the culturing is performed at a temperature of 31° C.
65. The method of clause 1, any other suitable clause, or any combination of suitable clauses, wherein the culturing is performed at a temperature of 32° C.
66. The method of clause 1, any other suitable clause, or any combination of suitable clauses, wherein the culturing is performed at a temperature of 33° C.
67. The method of clause 1, any other suitable clause, or any combination of suitable clauses, wherein the culturing is performed at a temperature of 34° C.
68. The method of clause 1, any other suitable clause, or any combination of suitable clauses, wherein the culturing is performed at a temperature of 35° C.
69. The method of clause 1, any other suitable clause, or any combination of suitable clauses, wherein the culturing is performed at a temperature of 36° C.
70. The method of clause 1, any other suitable clause, or any combination of suitable clauses, wherein the culturing is performed at a temperature of 37° C.
71. The method of clause 1, any other suitable clause, or any combination of suitable clauses, wherein the culturing is performed at a temperature of 38° C.
72. The method of clause 1, any other suitable clause, or any combination of suitable clauses, wherein the culturing is performed at a temperature of 39° C.
73. The method of clause 1, any other suitable clause, or any combination of suitable clauses, wherein the culturing is performed at a temperature of 40° C.
74. The method of clause 1, any other suitable clause, or any combination of suitable clauses, wherein the culturing is performed at a global oxygen delivery between 25% and 75%.
75. The method of clause 1, any other suitable clause, or any combination of suitable clauses, wherein the culturing is performed at a global oxygen delivery between 25% and 50%.
76. The method of clause 1, any other suitable clause, or any combination of suitable clauses, wherein the culturing is performed at a global oxygen delivery between 35% and 50%.
77. The method of clause 1, any other suitable clause, or any combination of suitable clauses, wherein the culturing is performed at a global oxygen delivery of 25%.
78. The method of clause 1, any other suitable clause, or any combination of suitable clauses, wherein the culturing is performed at a global oxygen delivery of 30%.
79. The method of clause 1, any other suitable clause, or any combination of suitable clauses, wherein the culturing is performed at a global oxygen delivery of 35%.
80. The method of clause 1, any other suitable clause, or any combination of suitable clauses, wherein the culturing is performed at a global oxygen delivery of 40%.
81. The method of clause 1, any other suitable clause, or any combination of suitable clauses, wherein the culturing is performed at a global oxygen delivery of 45%.
82. The method of clause 1, any other suitable clause, or any combination of suitable clauses, wherein the culturing is performed at a global oxygen delivery of 50%.
83. The method of clause 1, any other suitable clause, or any combination of suitable clauses, wherein the culturing is performed at a global oxygen delivery of 55%.
84. The method of clause 1, any other suitable clause, or any combination of suitable clauses, wherein the culturing is performed at a global oxygen delivery of 60%.
85. The method of clause 1, any other suitable clause, or any combination of suitable clauses, wherein the culturing is performed at a global oxygen delivery of 65%.
86. The method of clause 1, any other suitable clause, or any combination of suitable clauses, wherein the culturing is performed at a global oxygen delivery of 70%.
87. The method of clause 1, any other suitable clause, or any combination of suitable clauses, wherein the culturing is performed at a global oxygen delivery of 75%.
88. The method of clause 1, any other suitable clause, or any combination of suitable clauses, wherein the culturing is performed at an air oxygen flow of 1 SLPH.
89. The method of clause 1, any other suitable clause, or any combination of suitable clauses, wherein the culturing is performed at an agitation between 50 rpm and 150 rpm.
90. The method of clause 1, any other suitable clause, or any combination of suitable clauses, wherein the culturing is performed at an agitation between 60 rpm and 120 rpm.
91. The method of clause 1, any other suitable clause, or any combination of suitable clauses, wherein the culturing is performed at an agitation between 60 rpm and 90 rpm.
92. The method of clause 1, any other suitable clause, or any combination of suitable clauses, wherein the culturing is performed at an agitation between 90 rpm and 120 rpm.
93. The method of clause 1, any other suitable clause, or any combination of suitable clauses, wherein the culturing is performed at an agitation of 50 rpm.
94. The method of clause 1, any other suitable clause, or any combination of suitable clauses, wherein the culturing is performed at an agitation of 60 rpm.
95. The method of clause 1, any other suitable clause, or any combination of suitable clauses, wherein the culturing is performed at an agitation of 70 rpm.
96. The method of clause 1, any other suitable clause, or any combination of suitable clauses, wherein the culturing is performed at an agitation of 80 rpm.
97. The method of clause 1, any other suitable clause, or any combination of suitable clauses, wherein the culturing is performed at an agitation of 90 rpm.
98. The method of clause 1, any other suitable clause, or any combination of suitable clauses, wherein the culturing is performed at an agitation of 100 rpm.
99. The method of clause 1, any other suitable clause, or any combination of suitable clauses, wherein the culturing is performed at an agitation of 110 rpm.
100. The method of clause 1, any other suitable clause, or any combination of suitable clauses, wherein the culturing is performed at an agitation of 120 rpm.
101. The method of clause 1, any other suitable clause, or any combination of suitable clauses, wherein the culturing is performed at an agitation of 130 rpm.
102. The method of clause 1, any other suitable clause, or any combination of suitable clauses, wherein the culturing is performed at an agitation of 140 rpm.
103. The method of clause 1, any other suitable clause, or any combination of suitable clauses, wherein the culturing is performed at an agitation of 150 rpm.
104. The method of clause 1, any other suitable clause, or any combination of suitable clauses, wherein the culturing is performed at a seeding density of 0.4×10⁶ cells per mL.
105. The method of clause 1, any other suitable clause, or any combination of suitable clauses, wherein the culturing is performed at a seeding density of 0.5×10⁶ cells per mL.
106. The method of clause 1, any other suitable clause, or any combination of suitable clauses, wherein the culturing is performed at a seeding density of 0.6×10⁶ cells per mL.
107. The method of clause 1, any other suitable clause, or any combination of suitable clauses, wherein the culturing is performed at a seeding density of 0.7×10⁶ cells per mL.
108. The method of clause 1, any other suitable clause, or any combination of suitable clauses, wherein the culturing is performed at a seeding density of 0.8×10⁶ cells per mL.
109. The method of clause 1, any other suitable clause, or any combination of suitable clauses, wherein the culturing is performed at a seeding density of 0.9×10⁶ cells per mL.
110. The method of clause 1, any other suitable clause, or any combination of suitable clauses, wherein the culturing is performed at a seeding density of 1.0×10⁶ cells per mL.
111. The method of clause 1, any other suitable clause, or any combination of suitable clauses, wherein the cell culturing provides a viable cell density (VCD) above 98%.
112. The method of clause 1, any other suitable clause, or any combination of suitable clauses, wherein the cell culturing provides a viable cell density (VCD) above 95%.
113. The method of clause 1, any other suitable clause, or any combination of suitable clauses, wherein the cell culturing provides a viable cell density (VCD) above 90%.
114. The method of clause 1, any other suitable clause, or any combination of suitable clauses, wherein the cell culturing provides a viable cell density (VCD) above 85%.
115. The method of clause 1, any other suitable clause, or any combination of suitable clauses, wherein the cell culturing provides a viable cell density (VCD) above 80%.
116. The method of clause 1, any other suitable clause, or any combination of suitable clauses, wherein the cell culturing provides a viable cell density (VCD) above 75%.
117. The method of clause 1, any other suitable clause, or any combination of suitable clauses, wherein the cell culturing provides a viable cell density (VCD) above 70%.
118. The method of clause 1, any other suitable clause, or any combination of suitable clauses, wherein the cell culturing provides a viable cell density (VCD) above 65%.
119. The method of clause 1, any other suitable clause, or any combination of suitable clauses, wherein the cell culturing provides a viable cell density (VCD) above 60%.
120. The method of clause 1, any other suitable clause, or any combination of suitable clauses, wherein the cell culturing provides a viable cell density (VCD) above 55%.
121. The method of clause 1, any other suitable clause, or any combination of suitable clauses, wherein the cell culturing provides a viable cell density (VCD) above 50%.
122. The method of clause 1, any other suitable clause, or any combination of suitable clauses, wherein the cell culturing provides a viable cell density (VCD) above 45%.
123. The method of clause 1, any other suitable clause, or any combination of suitable clauses, wherein the cell culturing provides a viable cell density (VCD) above 40%.
124. The method of clause 11, any other suitable clause, or any combination of suitable clauses, wherein the method provides an increased expression level of the protein.
125. The method of clause 1, any other suitable clause, or any combination of suitable clauses, wherein the method provides an increased titer level of the protein.
126. The method of clause 1, any other suitable clause, or any combination of suitable clauses, wherein the method provides an increased amount of production of the protein.
127. A guanosine microparticle comprising a protein.
128. The guanosine microparticle of clause 127, any other suitable clause, or any combination of suitable clauses, wherein the protein is an HIV-1 envelope glycoprotein.
129. The guanosine microparticle of clause 127, any other suitable clause, or any combination of suitable clauses, wherein the protein is gp145.
130. The guanosine microparticle of clause 127, any other suitable clause, or any combination of suitable clauses, wherein the protein is encapsulated in the microparticle.
131, wherein the protein is presented on the outer surface of the microparticle.
132. The guanosine microparticle of clause 127, any other suitable clause, or any combination of suitable clauses, wherein the microparticle has a size between 1000 nm and 2000 nm.
133. The guanosine microparticle of clause 127, any other suitable clause, or any combination of suitable clauses, wherein the microparticle has a size between 1000 nm and 1100 nm.
134. The guanosine microparticle of clause 127, any other suitable clause, or any combination of suitable clauses, wherein the microparticle has a size between 1100 nm and 1200 nm.
135. The guanosine microparticle of clause 127, any other suitable clause, or any combination of suitable clauses, wherein the microparticle has a size between 1200 nm and 1300 nm.
136. The guanosine microparticle of clause 127, any other suitable clause, or any combination of suitable clauses, wherein the microparticle has a size between 1300 nm and 1400 nm.
137. The guanosine microparticle of clause 127, any other suitable clause, or any combination of suitable clauses, wherein the microparticle has a size between 1400 nm and 1500 nm.
138. The guanosine microparticle of clause 127, any other suitable clause, or any combination of suitable clauses, wherein the microparticle has a size between 1500 nm and 1600 nm.
139. The guanosine microparticle of clause 127, any other suitable clause, or any combination of suitable clauses, wherein the microparticle has a size between 1600 nm and 1700 nm.
140. The guanosine microparticle of clause 127, any other suitable clause, or any combination of suitable clauses, wherein the microparticle has a size between 1700 nm and 1800 nm.
141. The guanosine microparticle of clause 127, any other suitable clause, or any combination of suitable clauses, wherein the microparticle has a size between 1800 nm and 1900 nm.
142. The guanosine microparticle of clause 127, any other suitable clause, or any combination of suitable clauses, wherein the microparticle has a size between 1900 nm and 2000 nm.
143. The guanosine microparticle of clause 127, any other suitable clause, or any combination of suitable clauses, wherein the microparticle is an adjuvant.
144. The guanosine microparticle of clause 127, any other suitable clause, or any combination of suitable clauses, wherein the microparticle is configured for presentation of an antigen.
145. The guanosine microparticle of clause 127, any other suitable clause, or any combination of suitable clauses, wherein the microparticle is configured for delivery of a therapeutic agent.
146. The guanosine microparticle of clause 145, any other suitable clause, or any combination of suitable clauses, wherein the therapeutic agent is encapsulated in the microparticle.
147. A method of delivering a therapeutic agent to a patient, the method comprising the step of administering the guanosine microparticle of any one of claims xxx to the patient, wherein the microparticle comprises the therapeutic agent.
148. A method of presenting an antigen to a patient, the method comprising the step of administering the guanosine microparticle of any one of claims xxx to the patient, wherein the microparticle comprises the antigen.
149. A method of administering an adjuvant to a patient, the method comprising the step of administering the guanosine microparticle of any one of claims xxx to the patient, wherein the microparticle is the adjuvant.

### EXAMPLES

### Example 1

### Exemplary Experimental Procedures

The instant example provides exemplary materials and methods utilized in the examples as described herein.

### CHO-K1 Cell Line Expressing Gp145

A Chinese hamster ovary (CHO)-K1 cell line previously selected for the stable expression of uncleaved gp145 CO6980v0c22 was transferred from the Division of Acquired Immunodeficiency Syndrome (DAIDS), National Institute of Allergy and Infectious Diseases (NIAID), National Institutes of Health (material transfer agreement-MTA #980611). Briefly, the chemically defined PowerCHO-2 conditioned media, supplemented with glutamine (4 mM) and puromycin (5 μg/mL), was inoculated with=>4.6×10⁵ cells/mL in a 5 L bioreactor (4 L working volume). Cells were cultured at 37° C., pH 7.2, dO₂ 50%, with 120 rpm agitation. Cells were harvested on day 7 and gave a maximum titer of 40 mg/L.

### Upstream and Downstream Strategies to Increase Gp145 Titers

Cell culture. Generally, seeding train, supplement studies, and media screening were performed in shake flasks. Initial optimization of growth conditions were per-formed in 250 mL shake flasks. For these studies, CHO-K1 cells were grown in PowerCHO-2 serum-free media supplemented with 1× penicillin/streptomycin, Glu-taMAX (4 mM), and puromycin (5 μg/mL) at pH 7.2. The entire manufacturing process (upstream and downstream), as well as all analytical tests used to investigate gp145's quality attributes, resulted in the development of auditable standard operating procedures (SOPs).

Optimal pH determination. The pH of the cultures was optimized using the DASGIP parallel bioreactor system (Eppendorf). Briefly, each of the three small biore-actors was filled with 200 mL of PowerCHO-2 serum-free media (supplemented as described above). The pH of the media was adjusted by sparging with CO₂ at 0.6 SLPM prior to inoculation with 0.5×10⁶ cells/mL. The cells were harvested after 9 to 11 days of growth (n=2). Three pH set points were tested (pH 7.2, 7.0, and 6.8) and maintained with CO₂. The dO₂ set point was 35% and air and oxygen flow was 1 SLPH and the agitation rate was 90 rpm. A control shake flask (SF) was run in parallel in a 5% CO₂ incubator, with the same medium components, without pH adjustment.

Scale-up. The production of gp145 was scaled-up in a Finesse G3 Lab Bioprocess Controller (Thermo Scientific) using BioBLU 50-L single-use bioreactors (40 L working volume, Eppendorf). CHO-K1 cells were grown for 11 days in PowerCHO-2 serum-free media (supplemented as described above with puromycin (10 μg/mL)) at pH 6.8. Prior to inoculation, the pH of the media was adjusted by sparging with CO₂ at 0.6 SLPM. The agitation rate was set to 60 rpm and the air sparger at 1.5 SLPM. The dO₂ set point was 35% and antifoam was used at 0.01%. The seeding density was 0.8×10⁶ cells/ml from passage 17, based on studies that showed a seeding density of 0.8×10⁶ cells/mL produced higher titers (108 mg/L) than a seeding density of 0.5×10⁶ cells/mL (86 mg/L). The temperature was maintained using a heating blanket at 37° C. The daily samples were not clarified. Viable cell density and the percent viability were determined using a Cellometer Auto T4 cell counter (Nexcelom) by staining cells with trypan blue. The growth performance was further monitored using a Cedex Bio Analyzer (Roche) for the following metabolites: glucose, glutamate, glutamine, GlutaMAX, ammonia (NH₃), lactate, and lactate dehydrogenase (LDH).

Downstream. Harvested supernatants were subsequently clarified with SartoClear (10 g/L), sterile filtered, and then stored at −80° C. in 1 L aliquots. After thawing, the supernatant was concentrated using 100 kDa MWCO PES Vivaflow 200 System (Sartorius), then buffer-exchanged into binding buffer (25 mM Tris pH 8.0, 1 mM CaCl₂), and 1 mM MnCl₂). Salts were added just before loading to the lentil lectin column (5 ml bed volume, Cytiva). The flow-through was collected and reloaded. The eluate was concentrated and exchanged into the TOYOPEARL binding buffer (25 mM Tris, pH 8.0). Samples were loaded onto a 10 ml TOYOPEARL NH2-750F column and eluted using a salt gradient elution from 0 to 1M NaCl in 25 mM Tris, pH 8.0. The purified gp145 was buffer exchanged into 1×PBS. The purification was performed using the ÄKTA Avant 25 System (GE). The final purified gp145 was stored at −80° C. in sterile conical glass vials to prevent the glycoprotein loss that occurs in plastic tubes.

Feed strategies. Different feed strategies were explored using 1 L glass Finesse bioreactors to further optimize the production of gp145. Three feeding strategies were tested: first, two feeds were given on day 3 and day 6; second, a condition to maintain glucose levels between 2 g/L and 3 g/L with a feed supplemented with 50 mM GlutaMAX. For the last strategy, glucose and GlutaMAX were both monitored daily, and fed as necessary to maintain glucose levels between 4 and 6 g/L; third, maintaining glucose levels between 4 g/L and 6 g/L while replenishing GlutaMAX to 4 mM as needed. A control, batch reactor was included without subjecting to any feed strategy. All reactors were seeded with 0.8×10⁶ cells/mL in PowerCHO-2 serum-free medium supplemented with 1× penicillin/streptomycin, GlutaMAX (4 mM) and puromycin (10 μg/mL). The operational parameters were as follow: pH 6.8, 37° C., dO₂ 35%, air and oxygen flow 0.03 SLPM, and agitation 90 rpm.

### Titer Determination and Hydrophobic Footprint Analysis Using Reversed Phase High-Performance Liquid Chromatography (RP-HPLC)

Titers were determined as is typical. The titer value is the average of three technical replicates, and the error reported is the standard deviation. The hydrophobic footprint analysis was carried out using the same method as for titer determination, with a minor change, namely (time/% B): 0/30, 2/30, 3.5/65, 4/70, 5/75, 6/75, 6.5/95, 9.5/95, and 10/30. To dilute gp145 supernatant samples, 1× Dulbecco's phosphate buffered saline (DPBS) (Corning) was used, and standards were prepared using spent media. All standards and samples were run in triplicate. The tail factor and retention time errors reported are the standard deviations.

### Host Cell Protein (HCP) Determination and Binding Affinity Analysis Using Bio-Layer Interfrerometry

HCP quantification and gp145 binding studies were performed using the Octet® QKe (FortéBio) instrument. For HCP concentration determination, the anti-CHO host cell proteins (HCP) detection kit (FortéBio) was used, which utilizes the 3G anti-CHO HCP antibodies from Cygnus. The CHO HCP standard curve concentrations include 200, 100, 50, 25, 12.5, 6.25, and 3.13 ng/ml. The HCP values were determined at 4 different dilutions (1:5,000, 1:10,000, 1:15,000, and 1:20,000) to ensure linearity of the readings. Each dilution was read in triplicate. The reported values are an average of twelve readings. To challenge the HCP quantitation method (detection sensitivity=0.5 ng/mL, per manufacturer), the Day 4 sample was spiked with HCP (80 ng/ml), which resulted in a recovery of 98%. The Kp values for the gp145 protein were determined by loading the ligand, the 4E10 (3 μg/ml) broadly neutralizing antibody (Polymun Scientific), onto an anti-human IgG Fc capture (AHC) sensor (FortéBio, cat no. 18-5060). The serial dilutions of gp145 were used from 1-0.125 μM for curve fitting. The flow rate was 1200 rpm at 30° C. All buffers contained 0.5× kinetics buffer (cat no. 18-5032, FortéBio). Data were analyzed using the FortéBio Analysis 8.2 program to obtain the rate of association (kₐ), rate of dissociation (k_{d}), and dissociation constant (K_{D}). A reference sensor was used to correct for background drift.

### Sodium Dodecyl Sulfate-Polyacrylamide Gel Electrophoresis (SDS-PAGE)

The SDS-PAGE Mini-PROTEAN TGX precast 10% gel (Bio-Rad) was run under reducing conditions as recommended by the manufacturer. The gel was run for 40 min at 200 V in 1× Tris/Glycine/SDS running buffer (Bio-Rad) and stained with Coomassie Blue. A 4-15% gradient gel (Bio-Rad) was run for 30 min at 50 V and then 2.5 hours at 100 V at room temperature in 1× Tris/Glycine buffer for the native gel.

### MALDI-TOF Mass Spectrometry

Whole protein analysis. The gp145 whole protein was analyzed in linear positive mode on an AB Sciex 4800 Plus MALDI time of flight/time of flight (TOF)/TOF Analyzer and confirmed with a Shimadzu MALDI-TOF 7090 mass spectrometer. 2 μl of the desalted sample (2.2 mg/ml) was mixed with 10 μl of the following matrix solution before spotting onto the insert: α-cyano-4-hydroxycinnamic acid (10 mg/ml) (CHCA, Fluka), acetonitrile (30%), and trifluoroacetic acid (0.1%).

Peptide mass fingerprint analysis. For peptide mass fingerprinting, the gp145 protein was analyzed with and without PNGase F prior to trypsin digestion. The mMass program and the Mascot tool were used to determine peptide's identity.

Glycan analysis. PNGase F was used to release glycans in-gel for glycan analysis. The data presented were obtained using the 2′,4′,6′-trihydroxyacetophenone monohydrate (THAP) matrix, which could be used in either positive or negative mode and ionized the glycans better than super 2,5-dihydroxybenzoic acid (sDHB). Both matrices, THAP and sDHB, yielded similar results (data not shown). To increase the signal, the drops were spotted one after the other. The glycans were examined in both positive and negative reflector modes for neutral glycans and acidic glycans. An in-house Python program was developed to calculate the relative abundance of the glycans in the following manner in order to select the peaks of the mass spectra. First, the monoisotopic peaks were identified for the most prominent glycan peaks. Then, the other isotopic peaks were identified for each glycan (±1 Da, ±2 Da, etc.). Last, the ions for all peaks for one glycan were added. Finally, all the totals for all the glycans of interest were added to obtain the total ions count. The relative abundance was determined by dividing the total ions for a given glycan by the total ions value. The column graph and raw data images was prepared using GraphPad Prism (Version 6.07) and the program GlycoWorkbench. The values shown are the average of three readings and the error bar represents the standard error of the mean.

### Mass Analysis

The 2100 Bioanalyzer (Agilent Technologies) microchip capillary gel electrophoresis system was used because it is a quick method for precisely determining the mass of biomolecules. As recommended by the manufacturer, 2 μg of gp145 protein were labeled in a 5 μl reaction volume and 7 ng of gp145 were loaded into one well. Agilent's high-sensitivity protein 250 kit was employed. Data were analyzed using the 2100 Expert Software v B.02.08.SI648 (SR2). 1×DPBS was used as diluent and to fill empty microchip wells when necessary.

Glycoform Profiling by Imaged Capillary Isoelectric Focusing (cIEF)

Seventy micrograms of gp145 protein was mixed in 100 μl of cIEF solution urea (3M), methylcellulose (0.35%), Servalyte 2-11 (1.8%), Servalyte 3-7 (1.8%), and phosphoric acid (8 mM). The focusing was done in two steps; the first step was run for 1 min at 1500 V and the second step was run for 7 minutes at 3000 V. Each sample was run in triplicate. The iCE3 system (ProteinSimple) with the PrinCE autosampler was used at 10° C. The electropherogram was calibrated using the iCE3 21 CFR Part 11 software V4.1 (ProteinSimple) and plotted using GraphPad Prism (Version 6.07). For group analysis the pI ranges were defined as follows: Group I (3.68-5.45), Group II (5.45-6.25), and Group III (6.25-7.50). Values for group analysis are the average of three runs and the error reported is the standard deviation.

### Size Distribution Analysis by Dynamic Light Scattering (DLS)

The DLS analysis was carried out using Sarstedt cuvettes (reference number: 67.754) and the Zetasizer Nano Zs (model number ZEN3600, Malvern Panalytical). Samples were diluted to 0.15 mg/ml in 1×DPBS. After being spun, samples were loaded into the cuvette. ICN PBS tablet with a dispersant refractive index of 1.330, temperature of 25° C., and viscosity of 0.8882 was selected as the dispersant. For data analysis, the accompanying software was employed. The values reported are the average of three measurements and the error reported is the standard deviation.

### Secondary Structure Analysis by Circular Dichroism

Data were collected using a 1-mm Jasco cuvette and a J-1500 circular dichroism (CD) spectrometer (Jasco). To acquire data, the following parameters were used: CD scale=200 mdeg/1.0dOD; D.I.T=4 sec; bandwidth=1.0 nm; pitch=0.1 nm; and scanning speed=50 nm/min. In 0.1×DPBS, the gp145 glycoprotein was used at a concentration of 100 μg/ml. For data analysis, the accompanying software was used.

### Synthesis of Supramolecular Hacky Sacks (SHS)

All commercially available reagents were used as synthesized and provided by the manufacturers. riefly, samples, termed as Supramolecular G-quadruplexes (SGQ), were prepared by dissolving ImAG (0.650 mL, 5 mM) in potassium thiocyanate (KSCN) at 800 mM. The solid components were dissolved using phosphate buffer saline (PBS) (pH 7.4) in a 7-mL glass vial. The SGQ samples were sonicated for ~1 min and left equilibrating in the refrigerator overnight.

### Fluorescent Labeling of Gp145

The gp145 labeling and bioconjugation reaction was carried out in a 4° C. cold room according to the manufacturer's protocol (CAS #: 3326-32-7). For the reaction, the starting concentration of gp145 was 0.6 mg/mL. The following was the procedure: In a 4-mL borosilicate vial, 1 mL sodium carbonate buffer (0.1 M, pH 9) was mixed with 1 mL gp145. Fluorescein isothiocyanate (FITC) was used as a labeling agent in anhydrous dimethyl sulfoxide (DMSO) at a concentration of 1 mg/mL. 50 μL of FITC reagent was slowly added to each mL of protein sample using a mechanical micropipette. The reaction was stirred in the dark for 8 hours. Subsequently, the protein was purified by removing unconjugated FITC using size-exclusion chromatography (PD10 column) using 1×PBS (pH 7.4) as the eluent. One-milliliter fractions of FITC labeled gp145 were collected in borosilicate vials. Afterwards, the FITC-labeled protein fractions were pooled and concentrated by centrifugation using 10-mL AMICON filter tubes (30 kDa MWCO) spinning at 3,900 rpm for 30 min. The final concentration of FITC-labeled gp145 (gp145-FITC) was measured using UV-VIS spectrophotometry.

### Preparation of the Gp145-FITC-SHS Complex and VRC01-Alexa Fluor 647 Complex

A stock solution containing ImAG (5 mM) and KSCN (800 mM) in 1×PBS was made fresh and left at −20° C. for 1 day. A second solution, termed as SHS solution, containing ImAG (0.3 mM) and KSCN (52 mM) was prepared by diluting 100 μL of the stock solution in 1.650 mL of PBS at 40° C. Then, 50 μL of the SHS solution was added to a 2-mL borosilicate vial and mixed with 50 μL of gp145 (0.282 mg/mL) in triplicates. These samples were then left to incubate spinning (100 rpm) at 40° C. for 1 hour then examined by flow cytometry and confocal microscopy. The fluorescent gp120, HIV-1 env gp120, strain IIIB, recombinant (FITC) was from MyBioSource (cat. #MBS635758).

### Flow Cytometry

Flow cytometry measurements were made using a BD Acurri™ C6 Flow Cytometer (BD Sciences) with standard laser configuration (488 nm and 640 nm) and detectors (FL1 525/25, FL2 585/15, FL3 670 LP, and FL4 675/25). Samples were analyzed quickly after encapsulation protocol finished in the incubation vial.

### Confocal Imaging

Fluorescent images were captured at the Neuroimaging & Electrophysiology Facility in the Molecular Science Center. The microscope utilized is an inverted Nikon Eclipse Ti with a 100× oil immersion Plan Apo λ objective. Stimulation and acquisition were performed by a Nikon A1 Laser Scanning Confocal unit using (excitation/emission) 488 nm/525 nm for FITC, 640 nm/700 nm for Alexa-647, and a differential interference contrast (DIC) configuration for transmitted light. The pinhole aperture was open to 0.8 airy and intensity profile analysis was performed on NIS Elements Software V5.3.

### Example 2

### Optimizing the Expression of Gp145

The CHO-K1 cell line expressing secreted gp145 CO6980v0c22 was obtained. Before its arrival, this cell line was cultured in 5 L bioreactors containing PowerCHO-2 chemically defined media. The culture supernatants gave a titer of 40 mg/L as measured by ELISA. First, eight different media bases with various supplementations were examined (Table 1) and confirmed that PowerCHO-2 was the best media base to grow the CHO-K1 cell line. Transferring production from shake flasks to a bioreactor resulted in a substantial decrease in the gp145 production titer from 91 mg/L in shake flasks down to 47 mg/L in the bioreactor (Table 2). To overcome this, the media pH at the end of the SF runs was analyzed, and it was pH 6.8.

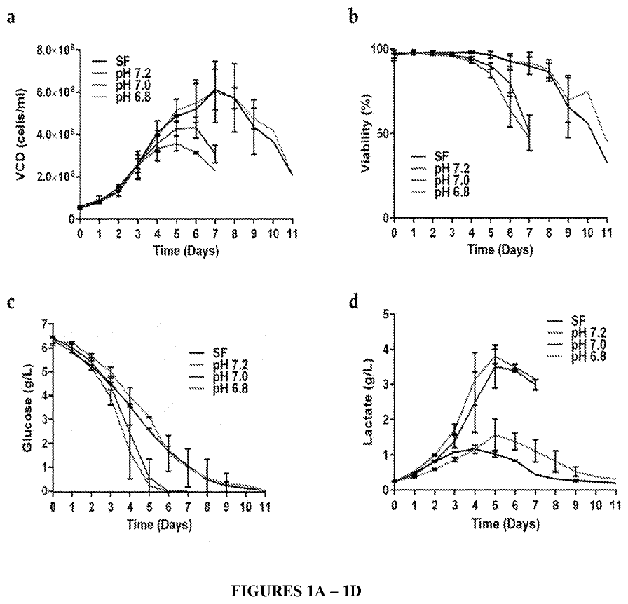

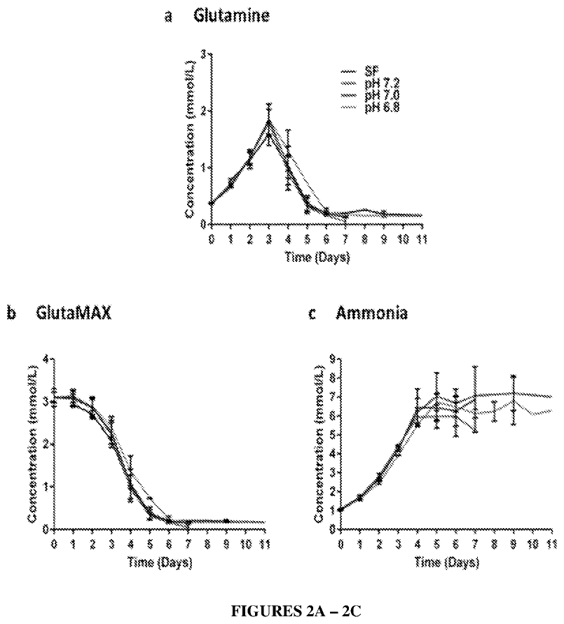

To confirm pH 6.8 as optimal for cell growth and gp145 production, small-scale bioreactor runs at different pH set points were performed. Results confirmed that, in bioreactors, a pH of 6.8 allows gp145 titers (86 mg/L) similar to those obtained with SF runs (91 mg/L) (Table 2). Since pH 6.8 resulted in the highest gp145 titer, this pH was used in subsequent runs. This change in pH set point allowed the cultures to remain productive for a longer time (FIGS. 1A and 1B), exhibiting viable cell density (VCD) values that remained above 80% during first 8 days and then VCD went down to 45% on day 11 when the harvest was made (FIG. 1A). These high VCDs observed during the first 8 days were accompanied by a high number of viable cells: 98.5 (day 1)-84.5% (day 8) (FIG. 1B), which eventually decreased without losing their productivity. This extension of cell culture time at pH 6.8 correlated with a prolonged decrease in glucose concentration in the bioreactor (FIG. 1C) together with a reduction of lactate accumulation after day 5 of culture (FIG. 1D). These data suggest that the CHO-K1 cells, at pH 6.8, are able to co-metabolize glucose and lactate during the growth phase at day 5, when lactate accumulation was reversed (FIG. 1D), and the VCD still had not reached its maximum cell density at day 7 (FIG. 1A). Monitoring of metabolites showed that from the third day, the glucose levels decreased constantly until almost completely consumed by day 7 in a process independent of the pH of the cell culture (FIG. 2A). This glucose consumption coincides with the decrease in the concentration of GlutaMAX (FIG. 2B) as well as with the accumulation of ammonia in it (FIG. 2C). From the fifth day, the accumulation of ammonia reached a plateau without significant changes until the eleventh day of culture monitoring (FIG. 2C).

TABLE 1
Cell culture media tested in examples.

| Media base tested | Estimated titer (μg/mL) | Comments |
|---|---|---|
| PowerCHO-1 | Not pursued | This culture medium was used to evaluate CHO- |
| | | K1 cells and adapt them to PowerCHO-2. |
| PowerCHO-2 | 160 | This was the culture medium that produced the |
| | | best titters and was used in our study |
| PowerCHO-3 | Not pursued | Not pursued, unavailable commercial or GMP- |
| | | grade |
| PowerCHO Advance | 45 | Not pursued due to low titers |
| OptiCHO | 158 | Not pursued due to high gp145 degradation |
| FortiCHO | 50 | Not pursued due to low titers |
| Dynamis | 50 | Not pursued due to low titers |
| BDCHO | 75-100 | Not pursued, unavailable commercial or GMP- |
| | | grade |

TABLE 2
pH studies using 0.250 L bioreactors

| pH | gp145 titer |
|---|---|
| setpoint | (mg/L) ± SD |
| 7.2 | 47 ± 0 |
| 7.0 | 59 ± 2 |
| 6.8 | 86 ± 8 |
| SF | 91 ± 11 |

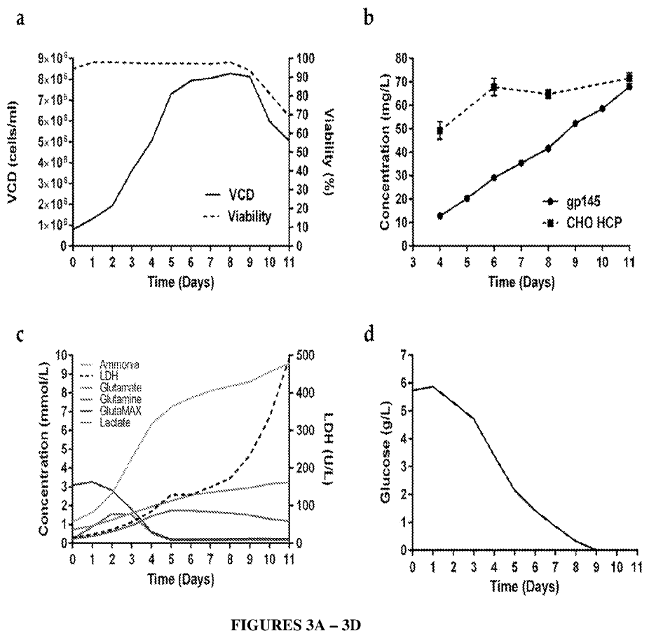

The production of gp145 was scaled up to a 50-liters bioreactor (40-L working volume) batch culture (FIG. 3A). Cell growth peaked at day 8 with a viable cell density of 8.3×10⁶ cells/ml and a viability of 98%. Although the cells began to decline in viability from day 9 onward, the growth was allowed to proceed since titers continued to increase without a substantial increase in the amount of CHO host cell proteins (HCPs) (FIG. 3B). During the 11 days of cell culture in this 50-L bioreactor, a time-dependent accumulation of ammonia was detected (FIG. 3C). Said accumulation did not reach the plateau observed in the 0.250 L bioreactor, instead it exhibited a sustained increase until harvest (day 11). For GlutaMAX, similar to what was observed in the 0.250 L bioreactor for day 5, the levels reached practically undetectable levels, demonstrating the consumption profile of the cells (FIG. 3C). Also, on day 5, lactate levels began to decline while lactate dehydrogenase (LDH) levels consistently spiked through harvest day. Glucose levels were depleted after 9 days (FIG. 3D). In theory, increasing the harvest time from 7 days to 11 days carries the risk of excessive cell death, potentially resulting in the release of large amounts of HCPs into the supernatant. The antibody-based biosensor assay showed that while the gp145 titers were increasing linearly until day 11, the abundance of HCPs in the supernatants remained relatively constant, further supporting the strategy of extending the lifetime of the culture (FIG. 3B). The clarified harvest sample gave a yield of 101 mg/L.

Supplementation of PowerCHO-2 with ornithine, citrulline, and chemically defined lipids showed no effect on titers (data not shown). To further optimize the production of gp145, the effect of continuous feed of nutrients as well as GlutaMAX supplementation was explored. In FIG. 3C, it was identified that both glutamine and GlutaMAX were limiting as the levels of both metabolites reached concentrations be-low detection limit after 5 days in culture while ammonia accumulation increased rapidly from day 2 and LDH production maintained an increasing rate from day 6. Glucose consumption remained constant until the glucose levels were below the detection limit. As shown in Table 3, exploration of the effect of adding GlutaMAX in the glucose feed strategy to supplement the culture under three conditions is shown. The objective of the first condition was to feed on particular days (day 3 and day 6) to replenish glucose to 6 g/L and GlutaMAX to 4 mM. The second condition included adding glucose and GlutaMAX in combination to maintain glucose levels between 2-3 g/L, with feedings on days 5, 7, and 11. The objective of the latter, was to maintain those levels, not to feed on particular days. The third condition was to supplement with glucose up to 6 g/L. From the results obtained, it is evident that the second treated condition, which included maintaining glucose levels between 2-3 g/L, produced the best titers (Table 3).

TABLE 3
Feed experiments to increase gp145 titer

| | | gp145 Titer |
|---|---|---|
| Condition # | Condition | (mg/L) ± SD |
| 1 | Glucose and GlutaMAX | 123 ± 2 |
| | (feed on day 3 and day 6) | |
| 2 | Glucose and GlutaMAX | 160 ± 1 |
| | feed (glucose between 2-3 | |
| | g/L) | |
| 3 | Glucose feed up to 6 g/L | 121 ± 1 |
| N/A | No Feed (control) | 109 ± 1 |

### Example 3

### Analysis of Whole Gp145 by SDS PAGE and MALDI-TOF Mass Spectrometry

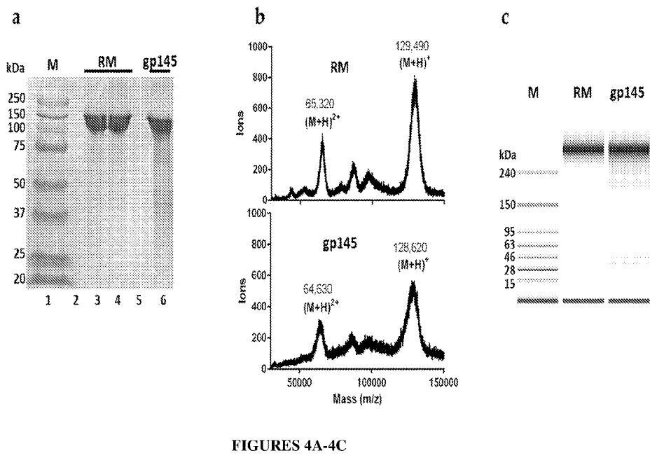

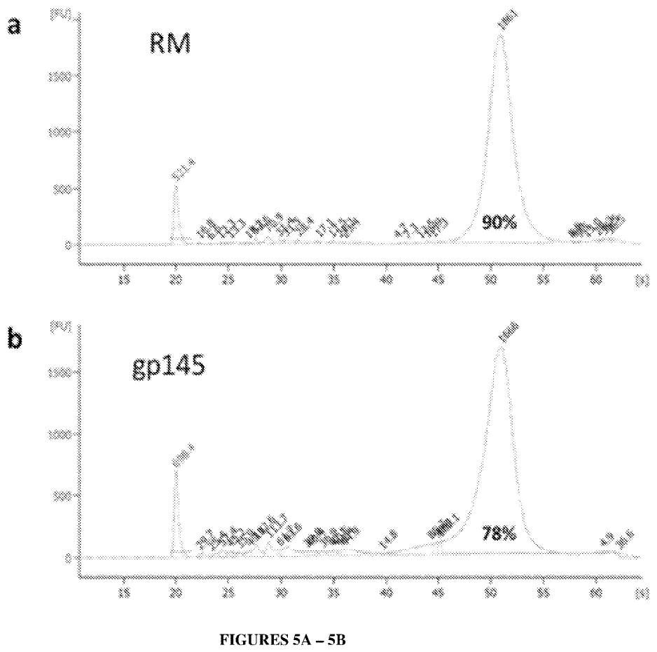

The yield of the purified gp145 was 1 mg from 1 L of supernatant. To confirm that the gp145 produced was similar in terms of molecular weight to the reference standard (RM), SDS-PAGE analysis was performed. The purified gp145 ran as expected, with a band corresponding to a 145 kDa protein (FIG. 4A). A complementary approach for the determination of the molar mass of gp145 was MALDI-TOF mass spectrometry of the intact full-length protein (FIG. 4B). Results showed a molar mass of 128,600 for the (M⁺H)⁺ ion with an additional signal of 64,600 corresponding to the (M⁺²H)²⁺ ion, similar to other published data. According to this mass spectrometry result, the contribution of the glycan shield to the molar mass of gp145 is 50,000 Da or roughly 40% of the total mass. A third method employed for the mass analysis of gp145 was the microchip capillary gel electrophoresis (MCGE) system, which provides higher sensitivity than traditional gel electrophoresis. MCGE analysis showed that gp145 migrates as a 310±5 kDa protein, which is outside of the linear range for the method, which has an upper limit of 250 kDa (FIG. 4C). The substantial decrease in the electrophoretic mobility observed by MCGE has been documented for other extensively glycosylated proteins. Although the exact causes for this electrophoretic delay are not known, it is thought to originate from a lower overall protein charge, since the extensive glycan shell on the protein limits its covering with detergent molecules. Nonetheless, this method can still be utilized to determine purity of the samples (FIG. 5A and FIG. 5B).

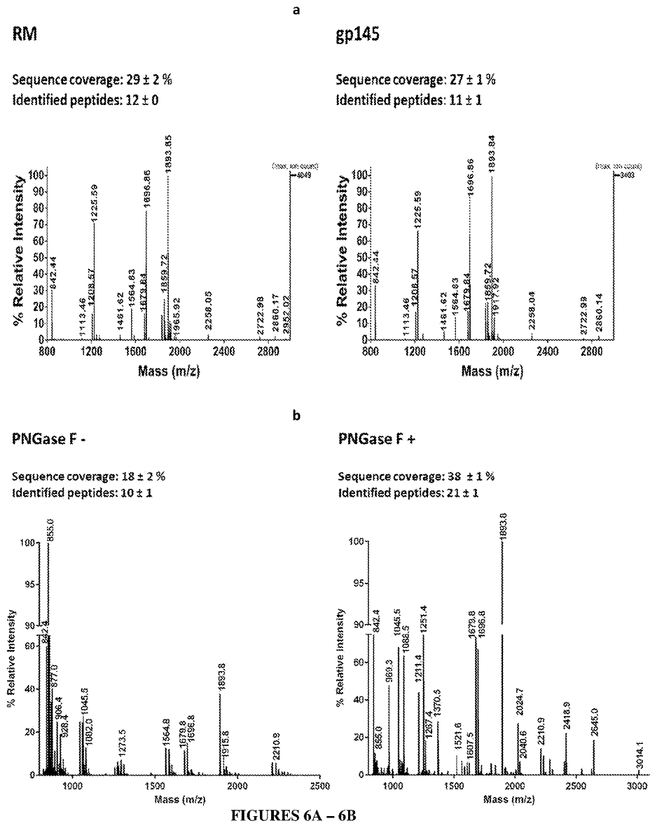

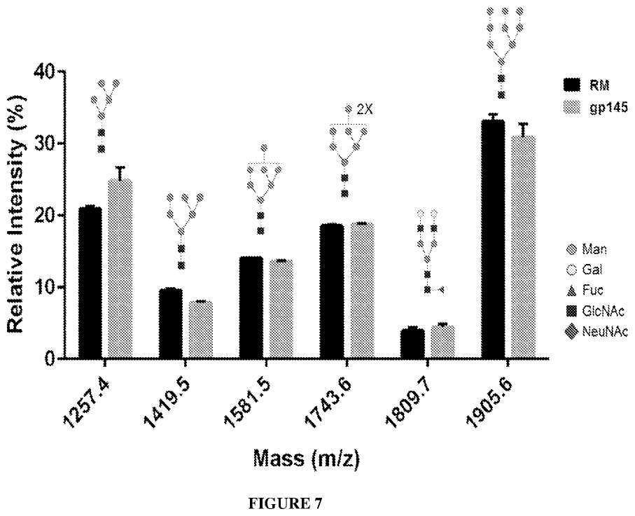

These methods clearly show that the purified gp145 reported here has similar attributes to the reference standard. Furthermore, both materials were confirmed to be the same amino acid sequence by peptide mass fingerprinting (PMF) as shown in FIG. 6A.

### Example 4

### Glycan Analysis

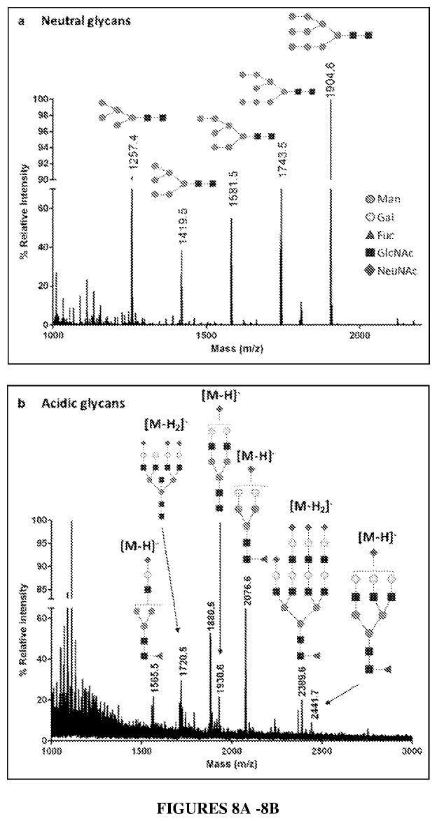

To show that the parameters adopted to increase gp145 titers did not affect the overall N-glycosylation profile of the vaccine candidate, glycan analysis by MALDI-TOF mass spectrometry was performed. First, gp145 was treated with PNGase F to determine how the removal of glycans affects the peptide mass fingerprinting. Removing the N-glycans with PNGase F resulted in a 2-fold increase in peptide coverage (from 18% to 38%), mostly due to an increased abundance of unmodified peptides in the analysis (FIG. 6B). Then, the gp145 PNGase F-released glycans were analyzed by MALDI-TOF. The neutral glycan profile was similar to that typically seen (FIG. 7 and FIG. 8A). Likewise, the acidic glycan profile coincides with glycans previously identified by other groups for HIV Env (FIG. 8B). The glycosidic linkage structure of the most prominent peak, mass 2076.6, was confirmed by collision-induced dissociation (data not shown).

### Example 5

### Biochemical Analysis of Gp145 by Capillary Isoelectric Focusing (cIEF) and RP-HPLC

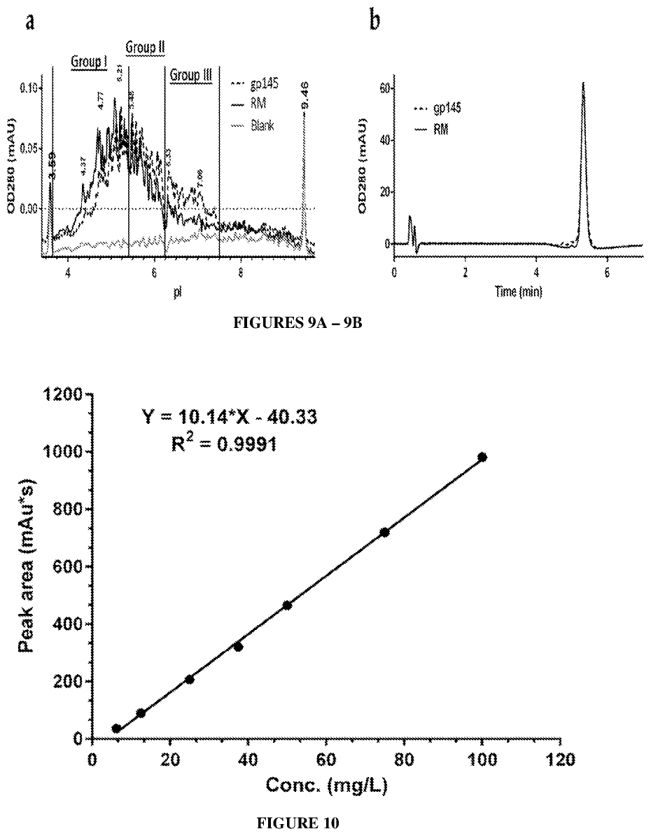

Although the predicted isoelectric point (pI) for gp145 polypeptide alone is above 7.0, the acidic glycans shift the gp145 μl and impart substantial charge heterogeneity. Analysis of the distribution of charged isoforms by capillary isoelectric focusing is shown in FIG. 9A. The pI distribution spans a range from 4.3 to 7.2, which is consistent with other pI determinations reported for gp120. Treatment of gp145 with neuraminidase A resulted in a substantially increased pI indicating that the native pI distribution is highly influenced by the presence of sialic acids. The pI distribution for the gp145 material made in the facility was very similar to that of the reference standard indicating similar levels of sialylation (Table 4). The RP-HPLC hydrophobic footprint analysis shows that the gp145 made in the UPR-CBC facility, is nearly identical to the RM (FIG. 9B). The retention times were identical for the purified gp145 and the reference standard, namely, 5.326±0.002 minutes. This retention time of less than 10 minutes was critical in developing a method that could be implemented into a quality assay. Furthermore, the tail factors were 1.008±0.005 for the in-house produced gp145 and 1.130±0.008 for the RM, suggesting a homogenous product and a peak shape consistent with the requirements for a quality control setting. The samples had a percent recovery of 106% and 107% for the RM and gp145, respectively; the standard curve used is shown in FIG. 10.

TABLE 4
cIEF percent area analysis

| | Group I (%) | Group II (%) | Group III (%) |
|---|---|---|---|
| RM | 30 ± 1 | 32 ± 1 | 39 ± 1 |
| gp145 | 31 ± 0 | 32 ± 0 | 37 ± 0 |

### Example 6

### Oligomeric State of Gp145

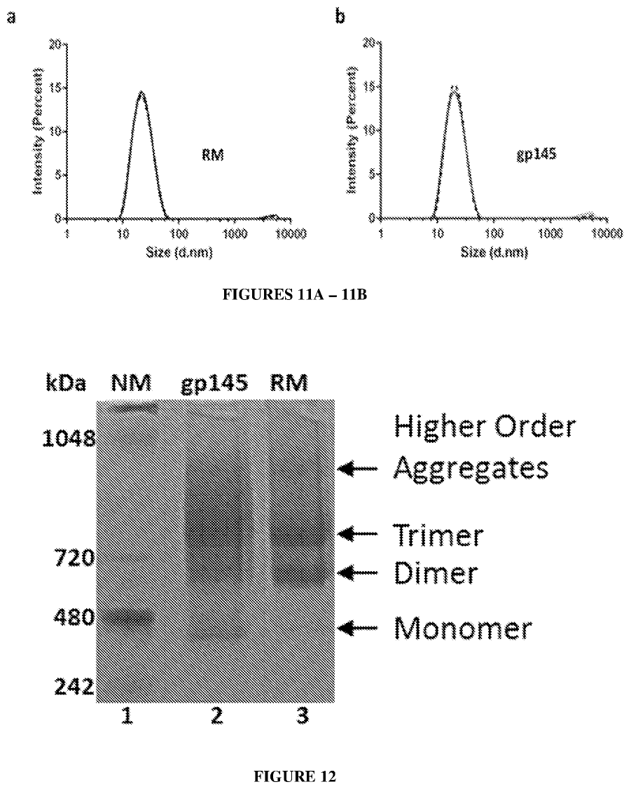

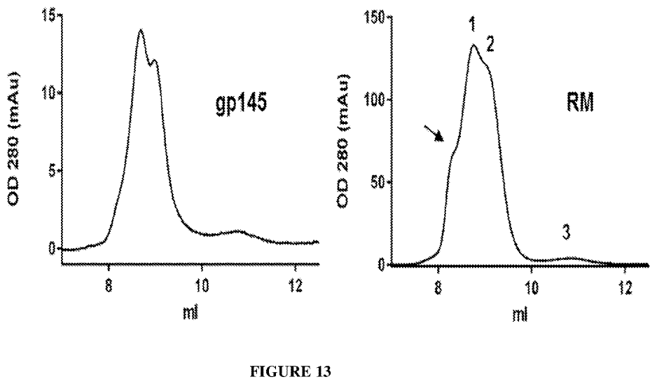

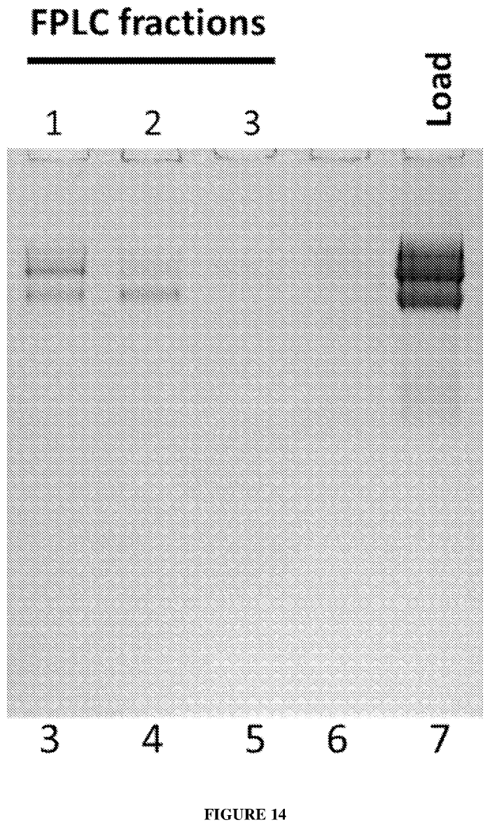

An important consideration in making new versions of Env longer than gp120 is the preservation of the native trimeric quaternary structure. Here it is reported by two different assays in which the oligomeric state of gp145 can be monitored. Dynamic light scattering (DLS) analysis shows that the gp145 protein is moderately (0.1-0.4) polydisperse because it has a polydispersity index (PDI) of 0.22 (Table 5, FIG. 11A and FIG. 11B), suggesting that it could be a mixture of different populations of gp145. This finding is consistent with the results obtained from native gel electrophoresis (FIG. 12), where four different populations of gp145 were observed, namely, higher order aggregates, trimer, dimer, and monomer. The observed distribution of trimers, dimers and monomers was the same as that reported previously. The assignment of bands was consistent with the negative stain EM images, revealing the true oligomeric states represented on the gel. Interestingly, gp145 produced in this manner seems to have less aggregate and more trimers than the reference standard. Size exclusion chromatography showed similar results (FIGS. 13 and 14).

TABLE 5
DLS size analysis of gp145

| | Z-avearge (d · nm) | PDI | % Intensity |
|---|---|---|---|
| RM | 23.5 ± 2.0 | 0.22 ± 0.06 | 93 ± 5 |
| gp145 | 21.5 ± 1.5 | 0.22 ± 0.05 | 95 ± 3 |

### Example 7

### Native-Like Properties of Gp145

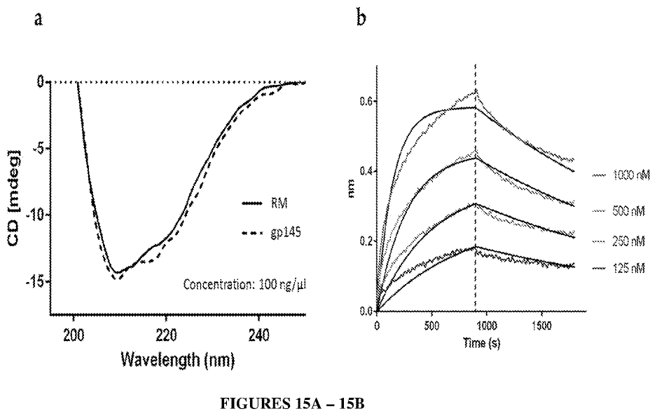

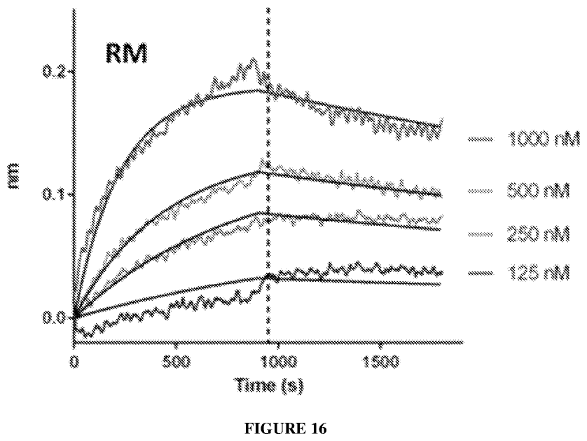

To assess the native-like properties of gp145, namely the secondary structure, the protein was analyzed by circular dichroism (CD). A broad absorption band with a minimum at 210 nm with an additional signal at 220 nm reveals the presence of a well-structured protein that is spectroscopically similar to the RM (FIG. 15A). Further confirmation of the native-like functionality of gp145 was evidenced by the binding affinity to the broadly neutralizing antibody 4E10, which was measured using biolayer interferometry (BLI) (FIG. 15B). The control for the experiment is presented in FIG. 16. The K_{D} values were 46 nM and 65 nM for the reference standard and for purified gp145 using this method, respectively (Table 6).

TABLE 6
Binding affinity analysis

| | K_{D} | Kₒₙ | K_{dis} | |
|---|---|---|---|---|
| | (nM) | (1/Ms) | (1/S) | Full R² |
| RM | 46 | 4.2E+03 | 2.0E−04 | 0.979 |
| gp145 | 65 | 6.5E+03 | 4.2E−04 | 0.979 |

### Example 8

### Encapsulation of gp145 for Delivery

In addition to the need for new immunogens, there is an increased drive to develop novel delivery platforms such as osmotic pumps and nanoparticles, to ensure a sustained release of immunogenic proteins. Toward this end, a self-assembling supramolecular hacky sacks (SHS) microparticles were tested for their ability to encapsulate the gp145. Previously, it was demonstrated that the SHS microparticles increased the frequency of IFN-γ-producing cells in a murine model independently immunized with pGag and pA27 L plasmids. The delivery of DNA plasmids coding “wild-type” antigens using these SHS microparticles efficiently increased antigen specific humoral and cell-mediated immunity in mice. To validate that these microparticles are capable of successfully encapsulating highly glycosylated proteins, FITC labeled gp120 (gp120-FITC) was encapsulated. Results obtained by flow cytometry show that the microparticles encapsulated gp120-FITC, a response that can be seen as a significant shift (96.4%) to the right in the cytometric histograms (FIG. 17A). The cytometric analysis focused on detecting FITC showed a significant increase (****P<0.0001) in the microparticles that incorporated gp120-FITC, compared to those not incubated with the protein (FIG. 17B). Similar responses were obtained for gp145-FITC. Thus, these SHS microparticles served to favorably encapsulate the highly glycosylated and heavy trimeric gp145. Said response can be appreciated by the notable displacement (68.3%) to the right in the histogram of the cytometric analysis (FIG. 17C). The encapsulation of gp145-FITC was statistically significant (****P<0.0001) (FIG. 17D). Altogether, the SHS microparticles prove to be a reliable vehicle for encapsulating and delivering the highly glycosylated antigen, gp145.

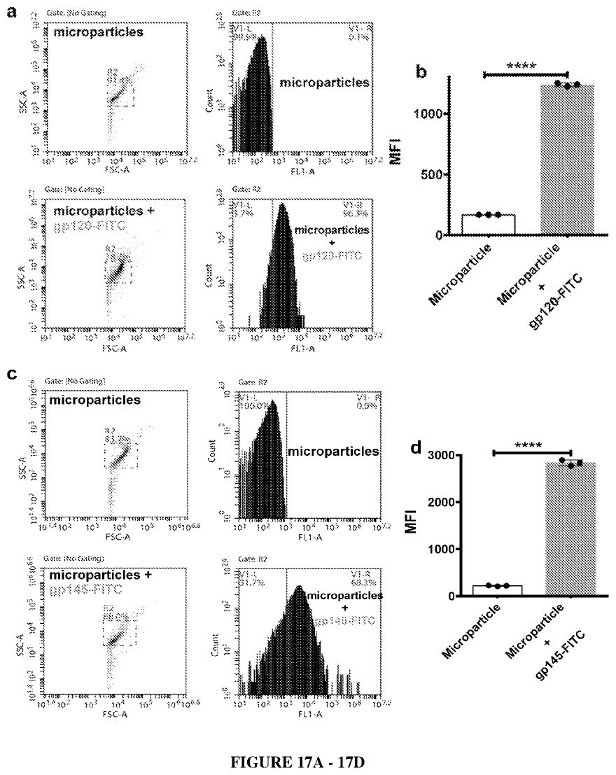

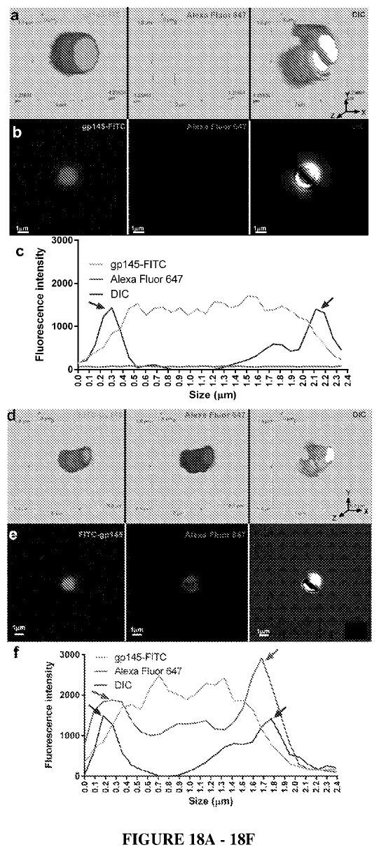

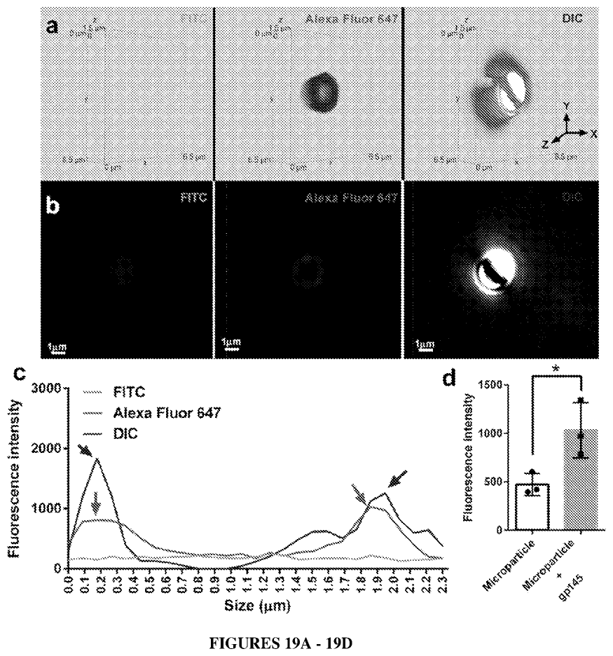

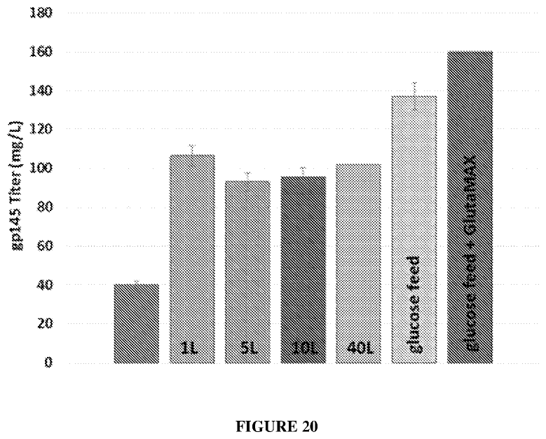

To understand the details of gp145-FITC encapsulation, SHS microparticles were examined by confocal microscopy. FIG. 18A shows 3D fluorescent and differential interference contrast (DIC) images of microparticles containing gp145-FITC (left) and microparticles alone (right) with a size of ~2000 nm (~2 μm). A 2D image shown was used to determine the distribution of the gp145-FITC envelope across the microparticles (FIG. 18B). The intensity of the gp145-FITC was homogeneously dispersed within microparticles (FIG. 18C). FIG. 18D shows two distinct microparticles of ~1600 nm, one loaded with gp145-FITC (left) and a second one that was first loaded with gp145-FITC followed by incubation with VRC01 broadly neutralizing antibody and subsequently incubated with a secondary antibody (Alexa Fluor 647) recognizing the VRC01 (acting as primary antibody). The broadly neutralizing antibody VRC01 binds strongly to monomeric gp120 and it is known that covers 98% of the target site, comprising 1089 Å² on the gp120 outer domain, about 50% larger than the 730 Å2 surface utilized by CD4. The 2D image shown in FIG. 18D was generated to determine the distribution of intensities of the gp145-FITC and Alexa Fluor 647-VRC01-gp145-FITC complex across the microparticle shown in FIG. 18E. The analysis of these images reveals that Alexa Fluor-647-VRC01-FITC-gp145 was distributed across the microparticles; however, unexpectedly, the highest intensities were located at the surface of these microparticles (the VRC01 binding towards the microparticle is negligible; see FIGS. 19A and 19B). FIG. 20 further summarizes the scaling process used in these examples, wherein the cell culture was successfully transferred to larger volume vessels employing various feeding strategies.

1. A method of producing a protein, the method comprising the steps of culturing one or more cells comprising the protein in a cell culture medium, wherein the cells express the protein, wherein the cell culture medium comprises glucose, glutamine, or a combination thereof.

2. The method of claim 1, wherein the cells secrete the protein into the cell culture medium.

3. (canceled)

4. (canceled)

5. The method of claim 1, wherein the protein is an HIV-1 envelope glycoprotein.

6. The method of claim 1, wherein the protein is gp145.

7. The method of claim 1, wherein the cells are Chinese Hamster Ovary (CHO) cells.

8. The method of claim 7, wherein the CHO cells are CHO-K1 cells.

9. The method of claim 7, wherein the CHO cells are engineered cells.

10. The method of claim 9, wherein the engineered cells express gp145.

11. The method of claim 10, wherein gp145 is uncleaved.

12. (canceled)

13. (canceled)

14. (canceled)

15. (canceled)

16. (canceled)

17. (canceled)

18. (canceled)

19. The method of claim 1, wherein the cell culture medium receives continuous feeding of glucose.

20. The method of claim 1, wherein the cell culture medium receives continuous feeding of glutamine.

21. The method of claim 1, wherein the culturing is performed at a pH between 6.0 and 8.0.

22. (canceled)

23. The method of claim 1, wherein the culturing is performed at a global oxygen delivery between 25% and 75%.

24. (canceled)

25. (canceled)

26. (canceled)

27. (canceled)

28. (canceled)

29. (canceled)

30. (canceled)

31. The method of claim 1, wherein the method provides an increased expression level of the protein.

32. The method of claim 1, wherein the method provides an increased titer level of the protein.

33. The method of claim 1, wherein the method provides an increased amount of production of the protein.

34. A guanosine microparticle comprising a protein, wherein the protein is gp145.

35. (canceled)

36. (canceled)

37. The guanosine microparticle of claim 34, wherein the protein is encapsulated in the microparticle.

38. The guanosine microparticle of claim 34, wherein the protein is presented on the outer surface of the microparticle.

39. The guanosine microparticle of claim 34, wherein the microparticle is configured for delivery of a therapeutic agent.
